(19) United States
(12) Patent Application Publication
(10) Pub. No.: US 2022/0185844 A1
(43) Pub. Date: Jun. 16, 2022

(54) PEPTIDE-DERIVED THERAPEUTICS TARGETING SET8 FOR THE TREATMENT OF CANCER

(71) Applicant: NUVOBIO CORPORATION, Ottawa (CA)

(72) Inventors: Kyle Kevin BIGGAR, Nepean (CA); Hemanta ADHIKARY, Nepean (CA)

(21) Appl. No.: 17/438,923

(22) PCT Filed: Mar. 11, 2020
(86) PCT No.: PCT/CA2020/050323

§ 371 Date: Sep. 13, 2021

Related U.S. Application Data
(60) Provisional application No. 62/818,251, filed on Mar. 14, 2019.

(51) Int. Cl. C07K 7/06; A61K 47/64; A61P 35/00; A61K 31/704

(52) U.S. Cl. CPC C07K 7/06; A61K 47/64; A61K 38/00; A61K 31/704; A61P 35/00

(57) ABSTRACT

The present invention relates to treatment of cancer. In particular, the present invention relates to peptides that bind SET8 for the treatment of cancer.

### FIELD OF THE INVENTION

The present invention relates to treatment of cancer. In particular, the present invention relates to peptide-derived therapeutics for the treatment of cancer.

### BACKGROUND

Two of every five Canadians are diagnosed with cancer at some point in their lives (Canadian Cancer Society, Cancer Statistics 2016). For the majority of cancers, targeted therapies are not yet available. For example, systemic chemotherapy is the only treatment option for triple negative breast cancer after surgery. However, chemotherapy is highly toxic and cancer cells can eventually become resistant to the treatment.

It is known that one gene mutation or one protein dysfunction does not initiate the development of cancer, but rather it is the dysregulation of a system of proteins that initiate the process and drives progression. As a result, there is an urgent need to understand the mechanism of cancer progression and chemoresistance in order to develop strategies to overcome resistance. Lysine methylation is essential in regulating many biological processes that range from growth and proliferation to pathological conditions, such as neurodegenerative disease, intellectual disability, and cancer. Given the extensive regulatory importance realized for lysine methylation, any mutations or dysfunction in methyltransferase (KMT) enzymes (i.e., the enzymes that catalyze the addition of lysine methylation) can lead to deregulated cell function, tumourigenesis and chemotherapy resistance (Arrowsmith et al., 2012; Hanamoto et al., 2015; Rao and Dou, 2015).

The realization that lysine methylation plays a critical role in the development of many human diseases is perhaps not a surprising one. It is well established that dynamic post-translational modifications (PTMs) made to protein, such as phosphorylation and methylation, play a crucial role in the transmission of biological signals (Seo and Lee, 2004; Beck-Sickinger and Mon, 2006; Zhang et al., 2012). These small chemical protein modifications allow for cells to exert greater control over specific cellular processes, while dysfunction within this PTM network are common drivers of cancer development and progression (Jin and Zangar, 2009). Dysfunction in the dynamic lysine methylation network (currently consisting of >5000 different lysine methylation modifications) has been identified as a prominent contributor to the development of many different types of cancer. For example, one pivotal study unveiled that the methyltransferase Set8 enhances the progression of bone and lung cancers through the dynamic methylation of p53 at lysine K382 (Shi et al., 2007). Given the involvement of lysine methylation in a growing number of different biological processes (Biggar and Li, 2015), KMT enzymes are emerging as a promising drug target.

To date, only a handful of KMT inhibitors have been discovered or developed, with almost all inhibitors currently within the preclinical stages of development (Hanamoto et al., 2015). Indeed, given the similarity between catalytic domains among families of KMT enzymes, it has been difficult to develop a small molecule inhibitor that is specific for a dysfunctional enzyme without significant off-target effects. Given the potential for substantial off-target toxicity, there is a critical need for more refined, enzyme-specific, inhibitors to be developed. Peptide-based therapeutics may be designed with exquisite specificity for their targets. This results in fewer side-effects from treatment. Peptide-based drugs also offer good efficacy, tolerability, predicted metabolism, lower attrition rates, and the advantage of a standard synthesis protocol.

### SUMMARY OF THE INVENTION

An object of the present invention is to provide peptide-derived therapeutics targeting Set8 for the treatment of cancer. In accordance with an aspect of the present invention, there is provided a peptide that binds to Set8.

In accordance with another aspect of the invention, there is provided a peptide that binds to Set8, wherein said peptide comprises the sequence:

X₁X₂X₃X₄X₅X₆X₇X₈X₉; where

### X₁=T or S

### X₂=H

### X₃=H

### X₄=H

### X₅=K or Nle

### X₆=H

### X₇=P

### X₈=G, H or E

X₉=Q, K, G, R, A, T or E; or a binding fragment thereof.

In accordance with another aspect of the invention, there is provided a peptide that binds to Set8 and comprises the sequence selected from the group consisting of THHHKHPHA; THHHKHPHH; THHHKHPKH; THHHKHPHT; THHHKHPKT; THHHK HPHK; THHHKHPEQ; THHHKHPHQ; SHHHKHPKA; THHHKHPHE; THHHKHPGT; THHHKHPEG; THHHKHPGH; THHHKHPHG; THHH KHPKA; THHHKHPGK; THHHKHPKK; THHHKHPGG; THHHKHPG Q; SHHHKHPGT; THHHKHPGR; THHHKHPGA; THHHKHPGE; SHH HKHPHG; SHHHKHPGH; THHHnHPHA; THHHnHPHH; THHHnHPK H; THHHnHPHT; THHHnHPKT; THHHnHPHK; THHHnHPEQ; THH HnHPHQ; SHHHnHPKA; THHHnHPHE; THHHnHPGT; THHHnHPE G; THHHnHPGH; THHHnHPHG; THHHnHPKA; THHHnHPGK; THHH nHPKK; THHHnHPGG; THHHnHPGQ; SHHHnHPGT; THHHnHPGR; THHHnHPGA; THHHnHPGE; SHHHnHPHG; andSHHHnHPGH; wherein n=norLeucine (Nle) or a binding fragment thereof.

In accordance with another aspect of the present invention, there is provided a peptide comprising the sequence selected from the group consisting of:

THHHnHPHQ{6-aminohexanoic acid}GRKKRRQRRRPPQ;
THHHKHPHK{6-aminohexanoic acid}GRKKRRQRRRPPQ;
THHHnHPGG{6-aminohexanoic acid}GRKKRRQRRRPPQ;
THHHKHPGR{6-aminohexanoic acid}GRKKRRQRRRPPQ;
THHHnHPGA{6-aminohexanoic acid}GRKKRRQRRRPPQ;
THHHKHPGA{6-aminohexanoic acid}GRKKRRQRRRPPQ;
SHHHnHPGT{6-aminohexanoic acid}GRKKRRQRRRPPQ;
THHHKHPGQ{6-aminohexanoic acid}GRKKRRQRRRPPQ;
THHHKHPGG{6-aminohexanoic acid}GRKKRRQRRRPPQ;
SHHHKHPHG{6-aminohexanoic acid}GRKKRRQRRRPPQ;
THHHnHPGE{6-aminohexanoic acid}GRKKRRQRRRPPQ;
SHHHnHPHG{6-aminohexanoic acid}GRKKRRQRRRPPQ;
THHHKHPEQ{6-aminohexanoic acid}GRKKRRQRRRPPQ;
THHHnHPEQ{6-aminohexanoic acid}GRKKRRQRRRPPQ;
THHHKHPHQ{6-aminohexanoic acid}GRKKRRQRRRPPQ;
THHHnHPHQ{6-aminohexanoic acid}FFLIPKGRRRRRRRRR;
THHHKHPHK{6-aminohexanoic acid}FFLIPKGRRRRRRRRR;
THHHnHPGG{6-aminohexanoic acid}FFLIPKGRRRRRRRRR;
THHHKHPGR{6-aminohexanoic acid}FFLIPKGRRRRRRRRR;
THHHnHPGA{6-aminohexanoic acid}FFLIPKGRRRRRRRRR;
THHHKHPGA{6-aminohexanoic acid}FFLIPKGRRRRRRRRR;
SHHHnHPGT{6-aminohexanoic acid}FFLIPKGRRRRRRRRR;
THHHKHPGQ{6-aminohexanoic acid}FFLIPKGRRRRRRRRR;
THHHKHPGG{6-aminohexanoic acid}FFLIPKGRRRRRRRRR;
SHHHKHPHG{6-aminohexanoic acid}FFLIPKGRRRRRRRRR;
THHHnHPGE{6-aminohexanoic acid}FFLIPKGRRRRRRRRR;
SHHHnHPHG{6-aminohexanoic acid}FFLIPKGRRRRRRRRR;
THHHKHPEQ{6-aminohexanoic acid}FFLIPKGRRRRRRRRR;
THHHnHPEQ{6-aminohexanoic acid}FFLIPKGRRRRRRRRR;
THHHKHPHQ{6-aminohexanoic acid}FFLIPKGRRRRRRRRR;
THHHnHPHQ{6-aminohexanoic acid}RRWRRWRRWRR;
THHHKHPHK{6-aminohexanoic acid}RRWRRWRRWRR;
THHHnHPGG{6-aminohexanoic acid}RRWRRWRRWRR;
THHHKHPGR{6-aminohexanoic acid}RRWRRWRRWRR;
THHHnHPGA{6-aminohexanoic acid}RRWRRWRRWRR;
THHHKHPGA{6-aminohexanoic acid}RRWRRWRRWRR;
SHHHnHPGT{6-aminohexanoic acid}RRWRRWRRWRR;
THHHKHPGQ{6-aminohexanoic acid}RRWRRWRRWRR;
THHHKHPGG{6-aminohexanoic acid}RRWRRWRRWRR;
SHHHKHPHG{6-aminohexanoic acid}RRWRRWRRWRR;
THHHnHPGE{6-aminohexanoic acid}RRWRRWRRWRR;
SHHHnHPHG{6-aminohexanoic acid}RRWRRWRRWRR;
THHHKHPEQ{6-aminohexanoic acid}RRWRRWRRWRR;
THHHnHPEQ{6-aminohexanoic acid}RRWRRWRRWRR;
and
THHHKHPHQ{6-aminohexanoic acid}RRWRRWRRWRR.

In accordance with another aspect of the invention, there is provided a peptide that binds to Set8, wherein said peptide comprises the sequence:

X₁=any amino acid

### X₂=H

### X₃=H

### X₄=H

### X₅=R, H, E, D, Q, K

### X₆=H, E

### X₇=P, N, Q, I, R, E

### X₈=M, N, E, D, Q, P, F, W, K, I, G, V

X₉=any amino acid, or a binding fragment thereof.

In other aspects of the present invention, there is provided methods of inhibiting the activity of Set8 in a subject in need thereof or methods of treating a disease associate with increased Set8, including but not limited to cancer, in a subject in need thereof, comprising administering one or more of the peptides of the invention.

### BRIEF DESCRIPTION OF THE FIGURES

FIG. 1. Array-based generation of Set8 binding peptides. (A) Oriented Peptide Array Library (OPAL) (top) followed by arrays of reduced amino acid degeneracy showcasing the systematic identification of peptide that display high affinity interaction with Set8. (B) Amino acid motifs corresponding to Set8 array binding. Motif images are generated from each neighboring array (left).

FIG. 2. Dose dependent inhibition of Set8 KMT activity in vitro. (A) Top Set8 inhibitors show significant inhibition of KMT activity in a dose-responsive manner. (B) The best performing inhibitor, KBL9 (EP18), inhibits Set8 activity in vitro. (C) KBL9 strongly associates with Set8. A Kd of 33.3+1-6.5 nM was determined for KBL9 using fluorescent polarization. (D) KBL9 was found to be specificity to Set8 among a panel of recombinant methyltransferase enzymes (Seth, Set7, Set8). * indicates significance at p<0.05. Average±SEM are shown (n=3, biological).

FIG. 3. Characterization of critical residues of the Set8 inhibitor, KBL9 by in vitro binding assay. (A) Progressive C-terminal, N-terminal, and tandem truncation of KBL9. Spot intensity (dark) indicates relative interaction with the SET domain of Set8 as detected by chemiluminescence. (B) Systematic mutation of the KBL9 alters Set8 binding activity. Relative binding preference of Set8 was systematically determined in order to assess the possible amino acid mutations of the KBL9 peptide that alter in vitro binding activity, either resulting in maintaining or strengthening (green), tolerable (yellow) or intolerable (red) Set8 SET domain interaction. WT KBL9 sequences are bolded. (C) Position-specific tolerable mutations (retaining>98% of WT binding) that can be made to KBL9 that allow for Set8 SET domain interaction.

FIG. 4. Delivery of Set8 inhibitor, KBL9, to colorectal carcinoma cell line. (A) Cell Viability assay with KBL9 conjugated with CPP (Mut6DPT), PR9, and TAT cell penetrating peptide. Cells treated with DMSO is considered as 100% active. Average±SD are shown (n=3, biological). (B) HCT 116 cells 24 hr post treatment with FITC-KBL9-TAT Set8 inhibitor stained with Hoechst nuclear stain, are shown under the fluorescent microscope in different channel viz., trans, Nuclei: Blue/Hoechst stain, FITC tagged peptides in green.

FIG. 5. Inhibition of Histone methylation levels by TAT-KBL9. (A) Western blot showing significant reduction of H4K20 mono-methylation level after treatment with 2, 5 and 10 nM of Set8 inhibitor TAT-KBL9. (B) Dose response after 24 hr post treatment with TAT-KBL9 in HEK 293 (non-cancer) and HCT 116 (colorectal carcinoma) cells. Average±SEM are shown (n=3, biological). (C) Scatter plot with bar graph depicting cell viability post 24 hr treatment with 20 nM KBL9.

FIG. 6. KBL9-TAT increases cell sensitivity to doxorubicin treatment. (A) HCT 116 cells were pre-treated with 2 uM KBL9-TAT for 24 hrs and then exposed to a dose-range of doxorubicin. Cell viability was determined by resazurin assay and normalized to TAT-alone control peptide. Scatter plot depicts average ±SEM (n=4, biological). (B) Area under the curve for dox-alone cells and cells pre-treated with KBL9-TAT. All data taken from panel A and are shown as increased dox response, relative to dox-alone treatment condition.

FIG. 7. Cell cycle distribution in KBL9-TAT treated HCT 116 cells. Two-parameter flow cytometric analysis of BrdU incorporation and DNA content was performed following a 24 hr exposure of TAT alone (A), 2 μM (B) and 5 μM (C) KBL9-TAT peptides to HCT 116 cells. (D) dose-response correlation of BrdU positive cells were represented. These values are expressed relative to untreated controls (+/−SEM) determined from 3 independent experiments.

### DETAILED DESCRIPTION

The present invention relates to peptide-derived therapeutics targeting enzymes in the lysine methylation pathway and the use of such therapeutics to treat diseases or disorders associated with dysfunction in lysine methylation. In particular, the present invention relates to peptide-derived therapeutics which target Set8 (also referred to as SetD8, KMTSA, histone H4K20me1 methyltransferase and EC 2.1.1.43) and the uses thereof.

### Peptides:

The present invention provides peptides that bind, optionally specifically bind, to Set8. In specific embodiments, the peptides of the present invention bind Set8 with high affinity. In specific embodiments, the peptides bind to Set8 and inhibit activity thereof. In certain embodiments, the peptides bind the catalytic core of Set8.

In certain embodiments of the present invention, the peptides comprise the consensus sequence (SEQ ID NO:71) set forth below

X₂X₃X₄nX₅X₆X₇X₈; where

### X₁=T or S

### X₂=H

### X₃=H

### X₄=H

n=K or Nle

### X₅=H

### X₇=G, H or E

### X₈=Q, K, G, R, A, T or E

Exemplary peptides are set forth in the table below:

| Experimental | SEQ | | Peptide |
|---|---|---|---|
| Peptide | ID NO: | Sequence | Name |
| EP 1 | 1 | THHHKHPHA | |
| EP 2 | 2 | THHHKHPHH | KBL6 |
| EP 3 | 3 | THHHKHPKH | |
| EP 4 | 4 | THHHKHPHT | |
| EP 5 | 5 | THHHKHPKT | |
| EP 6 | 6 | THHHKHPHK | KBL2 |
| EP 7 | 7 | THHHKHPEQ | KBL13 |
| EP 8 | 8 | THHHKHPHQ | KBL15 |
| EP 9 | 9 | SHHHKHPKA | |
| EP 10 | 10 | THHHKHPHE | |
| EP 11 | 11 | THHHKHPGT | |
| EP 12 | 12 | THHHKHPEG | |
| EP 13 | 13 | THHHKHPGH | |
| EP 14 | 14 | THHHKHPHG | |
| EP 15 | 15 | THHHKHPKA | |
| EP 16 | 16 | THHHKHPGK | |
| EP 17 | 17 | THHHKHPKK | |
| EP 18 | 18 | THHHKHPGG | KBL9 |
| EP 19 | 19 | THHHKHPGQ | KBL8 |
| EP 20 | 20 | SHHHKHPGT | |
| EP 21 | 21 | THHHKHPGR | KBL4 |
| EP 22 | 22 | THHHKHPGA | KBL6 |
| EP 23 | 23 | THHHKHPGE | |
| EP 24 | 24 | SHHHKHPHG | KBL10 |
| EP 25 | 25 | SHHHKHPGH | |
| EP 26 | 26 | THHHnHPHA | |
| EP 27 | 27 | THHHnHPHH | |
| EP 28 | 28 | THHHnHPKH | |
| EP 29 | 29 | THHHnHPHT | |
| EP 30 | 30 | THHHnHPKT | |
| EP 31 | 31 | THHHnHPHK | |
| EP 32 | 32 | THHHnHPEQ | KBL14 |
| EP 33 | 33 | THHHnHPHQ | KBL1 |
| EP 34 | 34 | SHHHnHPKA | |
| EP 35 | 35 | THHHnHPHE | |
| EP 36 | 36 | THHHnHPGT | |
| EP 37 | 37 | THHHnHPEG | |
| EP 38 | 38 | THHHnHPGH | |
| EP 39 | 39 | THHHnHPHG | |
| EP 40 | 40 | THHHnHPKA | |
| EP 41 | 41 | THHHnHPGK | |
| EP 42 | 42 | THHHnHPKK | |
| EP 43 | 43 | THHHnHPGG | KBL3 |
| EP 44 | 44 | THHHnHPGQ | |
| EP 45 | 45 | SHHHnHPGT | KBL7 |
| EP 46 | 46 | THHHnHPGR | |
| EP 47 | 47 | THHHnHPGA | KBL5 |
| EP 48 | 48 | THHHnHPGE | KBL11 |
| EP 49 | 49 | SHHHnHPHG | KBL12 |
| EP 50 | 50 | SHHHnHPGH | |

n = norLeucine (Nle)

Accordingly, in certain embodiments of the present invention, there is provided a peptide comprising the sequence as set forth in any one of SEQ ID NOs:1-50. In specific embodiments of the present invention, there is provided a peptide comprising any one of the sequences set forth in the table below:

| SEQ ID NO: | Sequence | Peptide Name |
|---|---|---|
| 2 | THHHKHPHH | KBL6 |
| 6 | THHHKHPHK | KBL2 |
| 7 | THHHKHPEQ | KBL13 |
| 8 | THHHKHPHQ | KBL15 |
| 18 | THHHKHPGG | KBL9 |
| 19 | THHHKHPGQ | KBL8 |
| 21 | THHHKHPGR | KBL4 |
| 22 | THHHKHPGA | KBL6 |
| 24 | SHHHKHPHG | KBL10 |
| 32 | THHHnHPEQ | KBL14 |
| 33 | THHHnHPHQ | KBL1 |
| 43 | THHHnHPGG | KBL3 |
| 45 | SHHHnHPGT | KBL7 |
| 47 | THHHnHPGA | KBL5 |
| 48 | THHHnHPGE | KBL11 |
| 49 | SHHHnHPHG | KBL12 |

n = norLeucine (Nle)

In certain embodiments of the present invention, there is provided peptides comprising variant sequences other than those specifically disclosed herein, which comprise significant sequence identity (e.g. 75%, 80%, 85%, 90%, 95%, 98%, or 99% sequence identity) to the amino acid sequence provided that such peptides retain the ability to inhibit Set8 activity. Such peptides can comprise one or more amino acid substitutions, additions, deletions, or insertions as compared to the parent amino acid sequence. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic amino acid substituted for another acidic amino acid (e.g. Asp or Glu), an amino acid with a nonpolar side chain substituted for another amino acid with a nonpolar side chain (e.g. Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc. In certain embodiments, naturally occurring amino acids in the peptides are replaced with amino acid analogs and derivatives thereof.

A worker skilled in the art could readily determine amino acid substitutions or truncations which impact binding activity of the peptides of the present invention. In certain embodiments, there is provided the KBL9 peptide (i.e. the peptide comprising THHHKHPGG) comprising one or more substitutions.

FIG. 3B provides details with respect to the impact of substitutions on the binding activity of KBL9. Following this systematic approach, position-specific tolerable mutations were identified. Accordingly, in certain embodiments, there is provided a peptide that binds to Set8, wherein said peptide comprises the sequence:

X₂X₃X₄X₅X₆X₇X₈X₉; where
X₁=any amino acid

### X₂=H

### X₃=H

### X₄=H

### X₅=R, H, E, D, Q, K

### X₆=H, E

### X₇=P, N, Q, I, R, E

### X₈=M, N, E, D, Q, P, F, W, K, I, G, V

X₉=any amino acid. (SEQ ID NO:72)

In certain embodiments of the present invention, there is provided peptides comprising a fragment of the sequences specifically disclosed herein comprising at least 5 contiguous amino acids, provided that such peptides retain the ability to inhibit Set8 activity.

In certain embodiments of the present invention, the peptides or fragments thereof comprise additional amino acids at the N and/or C terminus. In certain embodiments, the peptides of the present invention comprise A or AA at the N terminus. In certain embodiments, the peptides of the present invention comprise A or AA at the C terminus. In certain embodiments, the peptides of the present invention comprise A or AA at the N and C terminals. In certain embodiments of the present invention, there is provided a conjugate or fusion protein comprising the peptide of the present invention and heterologous amino acid sequence.

In certain embodiments, the peptides of the present invention includes a linker sequence. Linkers are known in the art and are generally classified into 3 categories according to their structures: (1) flexible linkers, (2) rigid linkers, and (3) in vivo cleavable linkers. Besides the basic role in linking the functional peptides together (as in flexible and rigid linkers) or releasing free functional peptide inhibitor in vivo (as in in vivo cleavable linkers), linkers may offer many other advantages for the production of inhibitor peptides, such as improving biological activity, increasing expression yield, and achieving desirable pharmacokinetic profiles.

| Linker | Model | Advantages | Example(s) |
|---|---|---|---|
| Flexible | [symbol] | Allows for interaction | (GGGGS)n, (G)n, |
| | | between functional peptide | 6-aminohexanoic |
| | | and delivery mechanism | acid (i.e., ahx) |
| | | (i.e., functional units) | |
| | [symbol] | Increases separation | |
| | | between functional units | |
| Rigid | [symbol] | Maintain distance | (EAAAK)n, (XP)n |
| | | between functional units | |
| Cleavable | [symbol] | Allows for in vivo | Disulphide, |
| | | separation of | protease |
| | | functional units | sensitive peptide |
| | | | sequences |

In certain embodiments, the peptides of the present invention further comprise a 6-aminohexanoic acid linker. The chemical structure of the linker is set forth below:

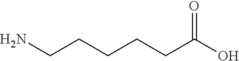

In specific embodiments, a cell penetrating peptide is conjugated to the peptide of the invention via a linker sequence.

In certain embodiments of the present invention, the peptides comprise other modifications including, without limitation, glycosylations, acetylations, phosphorylations, PEG, D-amino acids, nanoparticles, solid lipid nanoparticles, esterification, N-acetylation or may be formulated with liposomes, nano-emulsions, mucoadhesive polymers, nanoparticles, solid lipid nanoparticles.

It is known in the art that peptide modifications may improve therapeutic peptide delivery by increasing stability, inhibiting enzyme activity, enhancing absorption and/or cell targeting.

### Mechanisms of Therapeutic Peptide Delivery

| Goal | Peptide modification/formulations |
|---|---|
| Stomach | |
| Increased stability | PEG, D-amino acids, nanoparticles, solid lipid nanoparticles |
| Small intestine | |
| Increased stability | cyclization, PEG, lipidation, D-amino acids, polymer matrices, |
| | nanoparticles, esterification, N-acetylation |
| Enzyme inhibitors | soybean trypsin inhibitor, aprotinin, puromycin, bacitracin |
| Absorption enhancers | chitosans, fatty acids, lectins, Zonula occludens toxin, cell |
| | penetrating peptides, liposomes, nano-emulsions, |
| | mucoadhesive polymers, nanoparticles, solid lipid |
| | nanoparticles |
| Circulation | |
| Increased stability | PEG, hyper-glycosylation, liposomes, nanoparticles |
| Cell targeting | Antibody, cell penetrating peptides |

The peptides of the present invention may be coupled, either directly or via a linker, to a cell penetrating motif or other moiety so as to more efficiently facilitate the delivery of the peptide to the interior of a cell. Thus, the peptide can be provided as part of a composition or conjugate comprising the peptide and cell penetrating motif or other moiety. Any of various cell penetrating motifs and or other moieties useful for these purposes can be used. By way of illustration, suitable cell penetrating motifs and other relevant moieties (e.g. cell-membrane anchoring moieties) include lipids and fatty acids, cell penetrating peptides, and other types of carrier molecules (e.g. Pep-1).

In certain embodiments, the peptides of the present invention are coupled either directly or via a linker to a cell penetrating peptide. A repository of cell penetrating peptide can be found at crdd.osdd.net/Raghava/cppsite/index.html. Exemplary cell penetrating peptide are set forth in the table below:

| CPP name | Sequence | Origin | Class |
|---|---|---|---|
| TAT48-60 | GRKKRRQRRRPPQ (SEQ NO: 51) | HIV-1 TAT protein | Cationic |
| TAT49-57 | RKKRRQRRR (SEQ ID NO: 52) | HIV-1 TAT protein | Cationic |
| Penetratin, | RQIKIWFQNRRMKWKK (SEQ ID NO: 53) | Antennapedia Drosophila | Cationic |
| pAntp(43-58) | | melanogaster | |
| Polyarginines | Rn | Chemically synthesized | Cationic |
| DPV1047 | VKRGLKLRHVRPRVTRMDV (SEQ ID | Chemically synthesized | Cationic |
| | NO: 54) | | |
| PR9 | FFLIPKGRRRRRRRRR (SEQ ID NO: 55) | Chemically synthesized | Cationic |
| Mut6DPT (CPP) | RRWRRWRRWRR (SEQ ID NO: 56) | Chemically synthesized | Cationic |
| MPG | GALFLGFLGAAGSTMGAWSQPKKKRKV | HIV glycoprotein 41/SV40 T | Amphipathic |
| | (SEQ ID NO: 57) | antigen NLS | |
| Pep-1 | KETWWETWWTEWSQPKKKRKV (SEQ ID | Tryptophan-rich | Amphipathic |
| | NO: 58) | cluster/SV40 T antigen NLS | |
| pVEC | LLIILRRRIRKQAHAHSK (SEQ ID NO: 59) | Vascular endothelial | Amphipathic |
| | | cadherin | |
| ARF(1-22) | MVRRFLVTLRIRRACGPPRVRV (SEQ ID | p14ARF protein | Amphipathic |
| | NO: 60) | | |
| BPrPr(1-28) | MVKSKIGSWILVLFVAMWSDVGLCKKRP | N terminus of unprocessed | Amphipathic |
| | (SEQ ID NO: 61) | bovine prion protein | |
| MAP | KLALKLALKALKAALKLA (SEQ ID NO: 62) | Chemically synthesized | Amphipathic |
| Transportan | GWTLNSAGYLLGKINLKALAALAKKIL | Chimeric galanin- | Amphipathic |
| | (SEQ ID NO: 63) | mastoparan | |
| p28 | LSTAADMQGVVTDGMASGLDKDYLKPDD | Azurin | Amphipathic |
| | (SEQ ID NO: 64) | | |
| VT5 | DPKGDPKGVTVTVTVTVTGKGDPKPD | Chemically synthesized | Amphipathic |
| | (SEQ ID NO: 65) | | |
| Bac 7 | RRIRPRPPRLPRPRPRPLPFPRPG (SEQ | Bactenecin family of | Amphipathic |
| (Bac 1-24) | ID NO: 66) | antimicrobial peptides | |
| C105Y | CSIPPEVKFNKPFVYLI (SEQ ID NO: 67) | α1-Antitrypsin | Hydrophobic |
| PFVYLI | PFVYLI (SEQ ID NO: 68) | Derived from synthetic | Hydrophobic |
| | | C105Y | |
| Pep-7 | SDLWEMMMVSLACQY (SEQ ID NO: 69) | CHL8 peptide phage clone | Hydrophobic |

Repository can be found at crdd.osdd.net/Raghava/cppsite/index.html}

In certain embodiments of the present invention, a TAT cell penetrating peptide is linked either directly or via a linker to the peptides of the present invention. In certain embodiments of the present invention, a TAT cell penetrating peptide comprising the sequence GRKKRRQRRRPPQ (SEQ ID NO:51) is linked to the peptides of the present invention directly or via a linker. In certain embodiments of the present invention, a TAT cell penetrating peptide comprising the sequence GRKKRRQRRRPPQ is linked to the peptides of the present invention via a 6-aminohexanoic acid linker. In certain embodiments, the cell penetrating peptide is linked to the N-terminus of the peptide either directly or indirectly via a linker. In certain embodiments, the cell penetrating peptide is linked to the C-terminus of the peptide either directly or indirectly via a linker.

In specific embodiments of the present invention, there is provided a peptide-derived inhibitor comprising the sequence set forth in the table below:

| Inhibitor | Sequence |
|---|---|
| KBL9-TAT | THHHKHPGG{6-aminohexanoic acid} |
| | GRKKRRQRRRPPQ |
| KBL9-PR9 | THHHKHPGG{6-aminohexanoic acid} |
| | FFLIPKGRRRRRRRRR |
| KBL9-CPP | THHHKHPGG{6-aminohexanoic acid} |
| | RRWRRWRRWRR |

The peptides of the present invention can be biochemically synthesized such as by using standard solid phase techniques. These methods include exclusive solid phase synthesis, partial solid phase synthesis methods, fragment condensation, classical solution synthesis. Solid phase polypeptide synthesis procedures are well known in the art.

Recombinant techniques may also be used to generate the peptides of the present invention. Such recombinant techniques are known in the art. Accordingly, the present invention also provides a nucleic acid encoding the amino acid sequence of the peptide, and conjugates comprising the peptide. The nucleic acid can comprise DNA or RNA, and can be single or double stranded. Furthermore, the nucleic acid can comprise nucleotide analogues or derivatives (e.g. inosine or phophorothioate nucleotides and the like). The nucleic acid can encode the amino acid sequence of the peptide as part of a fusion protein comprising such sequence and a cell penetrating motif. The nucleic acid encoding the amino acid sequence of the peptide can be provided as part of a construct comprising the nucleic acid and elements that enable delivery of the nucleic acid to a cell, and/or expression of the nucleic acid in a cell. Such elements include, for example, expression vectors and transcription and/or translation sequences. Suitable vectors, transcription/translation sequences, and other elements, as well as methods of preparing such nucleic acids and constructs, are known in the art.

Accordingly, in certain embodiments polynucleotide encoding and expressing one or more peptide(s) of the invention. In another preferred embodiment, the polynucleotide is inserted in a vector. Preferably, said recombinant vector is an expression vector capable of expressing said polynucleotide when transfected or transformed into a host cell such as a prokaryotic or eukaryotic cell. The polynucleotide is inserted into an expression vector in proper orientation and correct reading frame for expression. In certain embodiments, the polynucleotide is operably linked to at least one transcriptional regulatory sequence and, optionally to at least one translational regulatory sequence. Recombinant vectors are known in the art and include but are not limited to plasmids and viral vectors. Viral vectors include but are not limited to oncolytic viral vectors, lentivirus and adenovirus vectors.

### Pharmaceutical Compositions:

The peptides and peptide derived inhibitors of the present invention be formulated as a pharmaceutical composition. In certain embodiments, the pharmaceutical composition comprises one or more peptides and peptide derived inhibitors of the invention alone or in combination with one or more other active agents and a pharmaceutically acceptable carrier.

Polynucleotides and vectors encoding the peptides of the invention may also be formulated as pharmaceutical compositions. In certain embodiments, the pharmaceutical composition comprises one or more polynucleotides or one or more vectors of the present invention alone or in combination with one or more other active agents and a pharmaceutically acceptable carrier.

The pharmaceutical composition may comprise one or more other pharmaceutically active agents or drugs. Examples of such other pharmaceutically active agents or drugs that may be suitable for use in the pharmaceutical composition include anticancer agents. Suitable anticancer agents include, without limitation, alkylating agents; nitrogen mustards; folate antagonists; purine antagonists; pyrimidine antagoinists; spindle poisons; topoisomerase inhibitors; apoptosis inducing agents; angiogenesis inhibitors; podophyllotoxins; nitrosoureas; cisplatin; carboplatin; interferon; asparginase; tamoxifen; leuprolide; flutamide; megestrol; mitomycin; bleomycin; doxorubicin; irinotecan; and taxol, geldanamycin and various anti-cancer peptides and antibodies.

The carrier may be any of those conventionally used and is limited only by physio-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. The pharmaceutically acceptable carrier may be one which is chemically inert to the active agent(s) and one which has no detrimental side effects or toxicity under the conditions of use.

The choice of carrier will be determined in part by the active agents, as well as by the method of administration. Accordingly, there are a variety of suitable formulations of the pharmaceutical composition of the present inventive methods. The following formulations for oral, aerosol, topical, parenteral, subcutaneous, intravenous, intramuscular, interperitoneal, rectal, and vaginal administration are exemplary and are in no way limiting. One skilled in the art will appreciate that these routes of administering the compound of the invention are known, and, formulations appropriate for each of these routes of administration are known in the art.

In certain embodiments, one or more peptides of the present invention are conjugated, directly or indirectly, to a carrier. Appropriate carriers are known in the art and include but are not limited to proteins including but not limited to keyhole limpet hemocyanin (KLH), bovine serum albumin (BSA) and ovalbumin (OVA); virus-like particles and viruses.

### Methods of Treatment

The present invention also provides methods of inhibiting Set8 activity. This method comprises bringing Set8 into contact with a peptide, peptide derived inhibitor or a pharmaceutical composition of the present invention. This contact may occur in vivo or in vitro. Accordingly, in certain embodiments, the present invention provides methods of inhibiting the activity of Set8 in a subject in need thereof, by administering one or more peptide(s), one or more peptide(s) derived inhibitor(s), one or more polynucleotide(s) or vector(s) encoding one or more peptide(s) or one or more pharmaceutical composition(s) of the present invention alone or in combination with one or more other active agents. The subject may be a mammal. In certain embodiments, the subject is a human.

The present invention also provides methods of treatment of disease associated with increased Set8 activity. Accordingly, in certain embodiments, the present invention provides methods of treatment of disease associated with increased Set8 activity in a subject in need thereof, by administering to the with one or more peptide(s), one or more peptide(s) derived inhibitor(s), one or more polynucleotide(s) or vector(s) encoding one or more peptide(s) or one or more pharmaceutical composition(s) of the present invention alone or in combination with one or more other active agents.

In certain embodiments, the disease associated with increased Set8 activity is a proliferative disease. In certain embodiments, the proliferative disease is cancer. Accordingly, in certain embodiments, the present invention provides methods of treatment of a cancer associated with increased Set8 activity in a subject in need thereof, by administering one or more peptide(s), one or more peptide(s) derived inhibitor(s), one or more polynucleotide(s) or vector(s) encoding one or more peptide(s) or one or more pharmaceutical composition(s) of the present invention alone or in combination with one or more other active agents.

The types of cancer include but are not limited to a cancer selected from the group consisting of acoustic neuroma; adenocarcinoma; adrenal gland cancer; anal cancer; angiosarcoma (e.g. lymphangiosarcoma, lymphangioendothelio sarcoma, hemangio sarcoma); appendix cancer; benign monoclonal gammopathy; biliary cancer (e.g. cholangiocarcinoma); bladder cancer; breast cancer (e.g. adenocarcinoma of the breast, papillary carcinoma of the breast, mammary cancer, medullary carcinoma of the breast, triple negative breast cancer (TNBC), ER positive breast cancer, ER negative breast cancer, PR positive breast cancer, PR negative breast cancer, ER/PR positive breast cancer, ER/PR negative breast cancer, HER2 positive breast cancer, HER2 negative breast cancer); brain cancer (e.g. meningioma, glioblastomas, glioma (e.g. astrocytoma, oligodendroglioma), medulloblastoma); bronchus cancer; carcinoid tumor; cervical cancer (e.g. cervical adenocarcinoma, squamous cell carcinoma of the cervix); choriocarcinoma; chordoma; craniopharyngioma; colorectal cancer (e.g. colon cancer, rectal cancer, colorectal adenocarcinoma); connective tissue cancer; epithelial carcinoma; ependymoma; endotheliosarcoma (e.g. Kaposi's sarcoma, multiple idiopathic hemorrhagic sarcoma); endometrial cancer (e.g. uterine cancer, uterine sarcoma); esophageal cancer (e.g. adenocarcinoma of the esophagus, Barrett's adenocarcinoma); Ewing's sarcoma; ocular cancer (e.g. intraocular melanoma, retinoblastoma); familiar hypereosinophilia; gall bladder cancer; gastric cancer (e.g. stomach adenocarcinoma); gastrointestinal stromal tumor (GIST); germ cell cancer; head and neck cancer (e.g. head and neck squamous cell carcinoma, oral cancer (e.g. oral squamous cell carcinoma), throat cancer (e.g. laryngeal cancer, pharyngeal cancer, nasopharyngeal cancer, oropharyngeal cancer)); heavy chain disease (e.g. alpha chain disease, gamma chain disease, mu chain disease; hemangioblastoma; hypopharynx cancer; inflammatory myofibroblastic tumors; immunocytic amyloidosis; kidney cancer (e.g. nephroblastoma a.k.a. Wilms' tumor, renal cell carcinoma); liver cancer (e.g. hepatocellular cancer (HCC), malignant hepatoma); lung cancer (e.g. bronchogenic carcinoma, small cell lung cancer (SCLC), non-small cell lung cancer (NSCLC), adenocarcinoma of the lung); leiomyosarcoma (LMS); mastocytosis (e.g. systemic mastocytosis); muscle cancer; myelodysplasia syndrome (MDS); mesothelioma; myeloproliferative disorder (MPD) (e.g. polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g. neurofibromatosis (NF) type 1 or type 2, schwannomatosis); neuroendocrine cancer (e.g. gastroenteropancreatic neuroendocrine tumor (GEP-NET), carcinoid tumor); osteosarcoma (e.g. bone cancer); ovarian cancer (e.g. cystadenocarcinoma, ovarian embryonal carcinoma, ovarian adenocarcinoma); papillary adenocarcinoma; pancreatic cancer (e.g. pancreatic adenocarcinoma, intraductal papillary mucinous neoplasm (IPMN), Islet cell tumors); penile cancer (e.g. Paget's disease of the penis and scrotum); pinealoma; primitive neuroectodermal tumor (PNT); plasma cell neoplasia; paraneoplastic syndromes; intraepithelial neoplasms; prostate cancer (e.g. prostate adenocarcinoma); rectal cancer; rhabdomyosarcoma; salivary gland cancer; skin cancer (e.g. squamous cell carcinoma (SCC), keratoacanthoma (KA), melanoma, basal cell carcinoma (BCC)); small bowel cancer (e.g. appendix cancer); soft tissue sarcoma (e.g. malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g. seminoma, testicular embryonal carcinoma); thyroid cancer (e.g. papillary carcinoma of the thyroid, papillary thyroid carcinoma (PTC), medullary thyroid cancer); urethral cancer; vaginal cancer; and vulvar cancer (e.g. Paget's disease of the vulva).

In specific embodiments of the present invention, there is provided a method of treatment of a cancer in a subject in need thereof, by administering one or more peptide(s), one or more peptide(s) derived inhibitor(s), one or more polynucleotide(s) or vector(s) encoding one or more peptide(s) or one or more pharmaceutical composition(s) of the present invention alone or in combination with one or more other active agents, wherein the cancer is selected from the group consisting of bladder, non-small lung carcinoma, small cell lung carcinoma, leukemia, liver, breast, colon, and pancreatic cancer.

In certain embodiments, the cancer is a metastatic cancer.

In certain embodiments, one or more peptide(s), one or more peptide(s) derived inhibitor(s), one or more polynucleotide(s) or vector(s) encoding one or more peptide(s) or one or more pharmaceutical composition(s) of the present invention are used in combination with additional pharmaceutical agents in the methods of the present invention.

The additional pharmaceutical agents may include but are not limited to anti-cancer agents. Anti-cancer agents encompass biotherapeutic anti-cancer agents as well as chemotherapeutic agents.

Exemplary biotherapeutic anti-cancer agents include, but are not limited to, interferons, cytokines (e.g. tumor necrosis factor, interferon a, interferon γ), vaccines, hematopoietic growth factors, monoclonal serotherapy, immuno stimulants and/or immunodulatory agents (e.g. IL-1, 2, 4, 6, or 12), immune cell growth factors (e.g. GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photo sensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated pro-drug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g. 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), antimetabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. l-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca<2+>ATPase inhibitors (e.g. thapsigargin), imatinib, thalidomide, lenalidomide, tyrosine kinase inhibitors (e.g. axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTEVIA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (V ARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g. bortezomib (VELCADE)), mTOR inhibitors (e.g. rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caraiinomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

### Example

The example below details the development of peptide inhibitors that target Set8 methyltrasferase (histone H4K20me1 methyltransferase). The focus on the Set8 methyltrasferase (also referred to as Set8 and histone H4K20me1 methyltransferase) is the result of its established role in cancer cell growth and proliferation, as well as its role in the coordination of chemotherapy-induced DNA damage. To date, Set8 has been found to be overexpressed in different types of cancer including bladder, non-small and small cell lung carcinoma, leukemia, liver, breast, colon, and pancreatic cancer (Dhami et al., 2013; Takawa et al., 2012). Although the exact mechanism of how this enzyme contributes to cancer progression has not yet been fully elucidated, it is believed that Set8 functions as an oncoprotein through the dynamic methylation (and functional regulation) of proteins with established roles in cancer biology. Indeed, as a consequence, the methyltransferase activity of the enzyme is implicated in many essential cellular processes including DNA replication, DNA damage response, transcription modulation, and cell cycle regulation. A primary example includes the Set8-mediated mono-methylation of p53 at lysine K382 (an important tumor-suppressor protein), suppressing p53 dependent transcriptional activation in cancer cells (Shi et al., 2007). Further implicating Set8 in p53 cancer biology, in models of breast cancer it has been shown that Set8 methylates a protein called NUMB at lysine(s) K158 and K163, decoupling its protective interaction with p53 and allowing p53 to undergo ubiquitination and degradation—effectively decreasing p53 activity and promoting cancer progression (Dhami et al., 2013). Set8 has also been shown to promote tumorigenesis through the methylation of PCNA at lysine K248 and cancer cell invasiveness and metastasis through its interaction with TWIST (Yang et al., 2012). Although these are only a few examples of Set8 function in cancer, an increasing number of studies are reporting on the key role played by Set8 in physiological and pathological pathways in the last decade (Guo et al., 2012; Song et al., 2009; Wang et al., 2012; Ding et al., 2012; Xu et al, 2013; Hashemi et al., 2014; Yao et al., 2014). Despite tremendous progress in the discovery of selective, small-molecule inhibitors of protein methyltransferases, only a limited number of inhibitors have been reported so far for Set8, with just a few of them being endowed with a certain degree of selectivity and/or cellular activity (Blum et al., 2014; Veschi et al., 2017). For example, the MC1947 and MC2569 inhibitors are two of only a few inhibitors with reported biological activity. In this regard, both inhibitors have been reported to reduce H4K20me1 and induce cellular death at dosages at (or above) 50 μM in U937 lymphoma cells (Valente et al., 2012).

### Materials and Methods

### Set8 Construct Information

The plasmid used to produce recombinant Set8 was:

pHIS2 Set8(191-352) Amp′

The sequence of recombinant Set8 is known in the art (Genes Dev. 2005 June 15:19(121:1455-651 and is set forth below:

(SEQ ID NO: 70)
AAIAKQALKKPIKGKQAPRKKAQGKTQQNRKLTDFYPVRRSSRKSKAEL
QSEERKRIDELIESGKEEGMKIDLIDGKGRGVIATKQFSRGDFVVEYHG
DLIEITDAKKREALYAQDPSTGCYMYYFQYLSKTYCVDATRETNRLGRL
INHSKCGNCQTKLHD

The crystal structure is also known in the art (DOI 10.2210/pdb1ZKK/pdb)

### Purification of Set8

Recombinant Set8-His6× were purified from Escherichia coli BL21 (DE3) RP strain. The cells were grown at 37° C. in 400 mL LB medium supplemented with 100 μg/ml ampicillin and 1 mM MgCl₂. At A₆₀₀=0.4, the culture was induced with 0.3 mM IPTG and incubated at 16° C. overnight (16 hr). The cells were harvested by centrifugation, washed with 1×PBS and the cell pellets were frozen in liquid nitrogen. For Set8-His6×, cells were lysed in P5 buffer (50 mM sodium phosphate, 500 mM NaCl, 10% glycerol, 0.05% triton X-100, 5 mM imidazole, pH 7) supplemented with protease inhibitors and homogenized by 20 passes through a dounce homogenizer (pestle A). The suspension was sonicated three times for 30 sec at 40% intensity. The cell lysate was then incubated with 1 mM MgCl₂ and 2.5 U/mL benzonase nuclease at 4° C. for 45 min followed by centrifugation at 18,000×g for 45 min using a Sorvall SS34 rotor. The soluble lysate was incubated with 400 μL HisPur™ Ni-NTA Resin (ThermoFisher Scientific; prewashed with P5 buffer) for 1 hr at 4° C. under gentle rotation. The Nickel resin was then washed three times (5 min each) with P30 buffer (P5 buffer with final concentration of 30 mM imidazole). Finally, the protein was eluted from the resin with 400 μL of P500 buffer (P5 buffer with a final concentration of 500 mM imidazole) for 5 min under rotation at 4° C., and the elution step was repeated 3 times to elute maximum and pure protein. The purified protein was dialyzed in storage buffer (20 mM Tris-HCl pH 7.5, 150 mM NaCl, 10% glycerol, 1 mM DTT), snap frozen on liquid nitrogen and stored in small aliquots at −80° C.

### Peptide Array Synthesis

The peptide libraries were synthesized on aminated cellulose membrane using the ResPep SL automatic peptide and SPOT array synthesizer (Intavis). An extra fine needle tip was used to achieve a density of 600 peptides per SPOT membrane (8×12 cm). Peptides were synthesized using fluorenylmethyloxycarbonyl chloride (Fmoc) chemistry and at a capacity of 2 nmol per array spot.

### Synthesis of Oriented Peptide Array Library (OPAL)

The peptide libraries were synthesized on aminated cellulose membrane using the ResPep SL automatic peptide and SPOT array synthesizer (Intavis). An extra fine needle tip was used to achieve a density of 600 peptides per SPOT membrane (8×12 cm). The following oriented peptide library arrays were synthesized for binding dependent interactions: AXXXX[Lys]XXXXA and AXXXX[nor-Leu]XXXXA; where X is a mixture of 19 amino acids (except Cys), and the brackets ([/]) encase the amino acids that were preferred by the protein of interest. To generate oriented peptide library pools, each degenerated position was scanned with any of the 19 amino acids (excluding Cys).

### Target Protein Binding Assays

The OPAL was designed sequentially starting from the most degenerate to highly specific peptide against our target protein as described above. The potential inhibitor peptides were initially screened based on the binding affinity between the peptides and target proteins. All the steps were carried out at room temperature unless otherwise stated. The OPAL cellulose macro arrays are presoaked in 100% ethanol followed by 50% ethanol for 15 min with constant rocking. The membrane is then washed with distilled water three times each of 15 min. The processed membrane is first blocked with 5% nonfat dry milk in Tris buffered saline containing 0.05% Tween 20 (TBST) for 1 hr at room temperature. Finally, the array was equilibrated with peptide binding buffer (50 mM Tris-CI, 350 mM NaCl, 10% glycerol, 0.5 mM DTT and 0.05% Tween20). The array was then incubated with 1 μM of target protein (Set8) overnight at 4° C. under rotation. The excess protein was washed away by three consecutive 10 min washes with TBST. Each array was then incubated with HRP conjugated anti-His antibody (1:5000) in TBST for 1 hr. The array was then washed thrice each of 10 min. The signals were detected using chemiluminescence. The signal intensities observed were subjected to densitometry analysis using ImageJ software protein array analyzer. Truncation and permutation peptide arrays designed to characterize the tolerability of KBL9 amino acid removal or substitution were processed under the same conditions as described above.

### Thermal Shift Using Differential Scanning Fluorimetry (DSF)

Thermal unfolding of target protein was monitored with inhibitor peptide using DSF. Optimal conditions were achieved using buffer containing 150 mM KH₂PO₄ (pH 7.5), 150 mM NaCl, 10 mM MgCl₂. To assess inhibitor-bound target protein (Set8), 20 μL/well (triplicate) reactions are set up into a Low 96-well Clear Multiplate PCR Plate (Bio-Rad). 125 μM of Set8 inhibitors and a DMSO control were briefly incubated with 20 μM Set8 in the optimized buffer. Sypro Orange™ at 5× (diluted in the above binding buffer from a stock concentration of 5000×) then added into the enzyme-peptide mixtures. The plate was covered with Microseal ‘B’ Film (Bio-Rad) and was equilibrated at 25° C. for 5 min. Experiments were conducted on a BioRad C1000 Thermal Cycler with CFX96 Real Time system. Heating was conducted using gradients from 25 to 95° C. (increasing 1° C. per minute). The remarkable shift in Tm were monitored at the end of cycling period.

### In Vitro Lysine Methyltransferase (KMT) Activity Inhibition Assay

Set8 KMT activity assays were performed using the bioluminescence-based Methyltransferase-Glo™ assay kit (Promega) according to manufacturer's instruction. Briefly, a substrate master mix was prepared with 10 μM H4K20 peptide and 10 μM S-adenosyl-L methionine (SAM) in methylation reaction buffer (20 mM Tris-HCl, pH 8, 3 mM MgCl₂, 50 mM NaCl, 1 mM EDTA, 1 mM DTT and 0.1 mg/mL BSA). Next, 4 μL of substrate mix was then aliquoted in wells of a white, solid bottom 384-well plates (Falcon). The reaction was then initiated by the addition of Set8 (50 nM) and inhibitor dilutions that were premixed in a total volume of 4 μL. Plates were then incubated for 30 min at room temperature. Upon completion, methyltransferase conversion of SAM to SAH was then detected using a two-step detection system where: 1 μL of MTase-Glo Reagent was added to each well to convert SAH to ADP for 30 min at room temperature. Finally, 5 μL of MTase-Glo Detection Solution was added to each well and allowed to incubate for 30 min at room temperature to convert ADP to ATP, which was then measured by luminescence detection using a ViewLux uHTS Microplate Imager (PerkinElmer) and compared to control samples to determine relative activity.

### Fluorescent Polarization

Recombinant Set8 protein was serially diluted in a 384-well plate, followed by the addition of fluorescein-labeled inhibitor peptide in PBS buffer. The mixtures were incubated in the dark for 30 min prior to fluorescent polarization measurements at room temperature on an EnVision Multilabel Plate Reader (PerkinElmer) with the excitation set at 480 nm and emission at 535 nm. Binding curves were generated by fitting the binding data to a hyperbolic nonlinear regression model using Prism 3.0 (GraphPad software, Inc., San Diego, Calif.), which also produced the corresponding dissociation constants (K_{d}).

### Delivery of Inhibitor Peptide to the Cell Line

Synthesis of three different cell penetrating peptide peptide with sequence Gly-Arg-Lys-Lys-Arg-Arg-Gln-Arg-Arg-Arg-Pro-Pro-Gln (i.e., TAT, SEQ ID NO:51), Phe-Phe-Leu-Ile-Pro-Lys-Gly-(Arg)₉ (i.e., PR9; SEQ ID NO:55) and (Arg-Arg-Trp)₃-Arg-Arg (i.e., MutD6 or CPP; SEQ ID NO:56) were carried out by solid phase synthesis on a ResPep SL peptide synthesizer (INTAVIS) following the Fmoc chemistry protocol. A 6-carboxyfluorescein (FITC derivative, referred to as only FITC in this document) was added to the C-terminal end of the peptides for ligation to a fluorochrome FITC and was separated by the addition of a 6-aminohexanoic acid group to provide both (1) fluor flexibility and (2) reduce steric constraints of the molecule.

To evaluate the internalization of FITC-labelled peptides, exponentially growing HCT 116 cells were seeded on 6 well plate at a density of 2×10⁵ cells per well and incubated overnight. After overnight incubation, the media (DMEM with pen-strep and 10% FBS) was replaced with fresh media supplemented with 10 μM FITC-labelled inhibitor peptides. Following the incubation (24 hr), cells were washed three times with ice cold PBS to remove the excess extracellular complexes. Cells were then stained with Hoechst dye (1:2000 dilution from 10 mg/mL stock in PBS) directly adding sufficient staining solution to the well. Cells were incubated for 10 min with the dye, protected from light. The staining solution is discarded, and the cells were washed 3 times with PBS and imaged directly under fluorescent microscope.

### Cell Viability Assay

Cell viability was measured using the Resazurin reduction assay which indirectly quantifies living cells through the metabolically active reduction of resazurin to fluorescent resorufin. This assay allows to maintain cells viability and, therefore, to monitor cell growth with time. Exponentially growing HCT 116 cells were seeded into 96-well plates at the density of 2.0×10⁴ cells/mL and incubated overnight. The media was replaced with fresh media prior to inhibitor treatment. Cells were treated with 10 μM of inhibitor peptide for 24 hr. All the inhibitors were diluted in the cell culture media (DMEM−/−) from 5 mM stock. All the treated cells were compared to the control (no treatment with equivalent quantity of DMSO) which were considered as 100% viable. One set of wells also prepared with medium only for background subtracting and instrument gain adjustment. The experiments were carried out in triplicate and expressed as mean+−SD. A 10% resazurin solution (0.15 mg/ml stock dissolved in PBS, filter sterilized and stored protected from light at 4° C.) was then added to each well and incubated for 2 hr. The fluorescence was recorded using a multiwell plate reader (Perkin Elmer) at Ex. 560 nm and Em. 610 nm.

The KBL9-TAT inhibitor was selected for further evaluation as it was found to demonstrate (1) high reproducibility and (2) maximum cell death from the above experiments and was subjected to dose to determine the LC₅₀ values in HCT 116 and HEK 293 cells. Briefly cells were treated with different concentrations of inhibitor for 24 hr ranging from 1 nM to 20 nM. The working dilutions of all the inhibitors were prepared in DMEM−/− media. As a non-cancerous cell model, HEK 293 cells were treated for 24 hr with KBL9-TAT inhibitor ranging from 1 nM to 20 nM to assess cell viability in comparison to HCT 116 carcinoma cells.

### Inhibition of Target Enzyme Activity in HCT 116 Cell Line

The topmost inhibitor, KBL9-TAT, from all the above experiments were tested for histone methylation status in HCT 116 cells. 3×10⁶ cells were plated in 10 cm dish and incubated overnight. Cells were then treated with the inhibitors at various concentrations (0, 0.5, 1, 2, 5, 10 nM). Cells (5×10⁶ cells/mL), 24 hr of post dosing, were collected in 15 mL falcon tube and centrifuged at 300×g for 10 min. The supernatant was discarded and the cells are washed with iced cold PBS. The cell pellet is flash-frozen in liquid nitrogen and stored at −80° C. Histone isolation is done using standard protocol. To summarize, cells were re-suspended in 1 mL hypotonic lysis buffer (10 mM Tris-HCl pH 8, 1 mM KCl, 1.5 mM MgCl₂ and 1 mM DTT) containing protease inhibitor. The cells were transferred to 1.5 mL tube and incubated for 30 min on rotor at 4° C. to promote hypotonic swelling and lysis. The intact nuclei are collected by centrifugation at 10,000×g for 10 min in a cooled tabletop centrifuge. The supernatant is entirely discarded and pellets were re-suspended completely in 600 μL 0.4N H₂SO₄ and incubated overnight on rotor at 4° C. The nuclear debris were removed by centrifugation at 16,000×g for 10 min. The supernatant containing the histones were transferred to a fresh 1.5 mL tube and precipitated by adding 195 μL TCA (33%) drop by drop. The reaction is incubated at 4° C. overnight under rotation. The histones were pelleted by centrifugation at 16,000×g for 10 min. After complete removal of the supernatant carefully, the histone pellets were washed with ice-cold acetone to remove the left-over acids without disturbing the pellet. Finally, the pellets were air dried for 30 min at room temperature. The histone pellets were dissolved in 100 μL milliQ water and stored frozen at −20° C. Samples of 1, 3 and 5 μL of histones were separated on 15% SDS-PAGE gel and stained with Coomassie Brilliant Blue and characterized on the quality and concentration of the histone. The locations of the linker histone H1 and the core histones H3, H2B, H2A and H4 were noted.

For western blot, of total of 1 μL of histones were separated on 15% SDS-PAGE and transferred overnight at 15V on PVDF membrane. Following blocking with 5% nonfat dry milk in 1×TBST for 1 hr, the membrane containing histones lanes treated with Set8 inhibitor, were probed with H4K20Me1 (Santa cruz) primary antibody (1:1000) in 1×TBST. Membranes were incubated overnight at 4° C. under rotation. Following this incubation, membranes were washed in 1×TBST for 30 min, followed by incubation with secondary antibody for an additional 1 hr. The membrane was then further washed for 30 min as before. Histone proteins were detected by Supersignal™ West Pico PLUS Chemiluminescent substrate (ThermoFisher Scientific) using the Chemidoc XRS+imaging system (BioRad).

### Flow Cytometry

A total of 0.3×10⁶ HCT 116 cells were plated in 6 well-plate and incubated overnight. Cells were treated with the inhibitors at various concentrations (0, 2, 5 μM), DMSO and cell penetrating peptide controls. For each condition, approximately 1×10⁶ HCT 116 cells were collected along with the floating cells in the media by centrifugation at 300×g for 10 min. The cells were then washed with 5 mL of ice-cold PBS and re-suspended in 0.5 mL of ice-cold PBS. The cells were slowly dropped into 4.5 mL of vortexing ice-cold 70% ethanol for rapid dispersion. The sample was incubated on ice for 45 min and then fixed at −20° C. overnight. The fixed cells were centrifuged at 4° C. at 300×g for 10 min. The resultant cell pellet was re-suspended to 200 μL of the stain master mix (133.7 μL of 1 mg/mL propidium iodide (PI), 1 μL of 10 mg/mL RNase A and PBS 865.3 μL). The PI-treated cells were incubated at 37° C. for 30 min and then analyzed by a flow cytometry (BD Accuri™ C6 Plus). The BD Accuri C6 Plus software version FCS 3.1 was used for apoptosis and cell cycle analysis.

### Chemosensitivity

Inhibitor-induced sensitivity of HCT 116 cells to a chemotherapeutic agent, namely doxorubicin (dox), was measured using the Resazurin reduction assay. This assay allows to maintain cells viability and, therefore, to monitor cell growth with time. Exponentially growing HCT 116 cells were seeded into 96-well plates at the density of 2.0×10⁴ cells/mL and incubated overnight. The media was replaced with fresh media prior to inhibitor treatment. Cells were pre-treated with 2 μM of inhibitor peptide for 24 hr. Peptide was diluted in the cell culture media (DMEM−/−) from 5 mM stock. All the treated cells were compared to the control (TAT peptide alone with equivalent quantity of DMSO) which were considered as 100% viable. One set of wells also prepared with medium only for background subtracting and instrument gain adjustment. Following 24 hr peptide treatment, dilutions of dox were added to cells (0, 0.001, 0.002, 0.01, 0.02, 0.1, 0.2, 1, 2 uM) and incubated for 16 hrs. The experiments were carried out in quadruplicate and expressed as mean+−SD. A 10% resazurin solution (0.15 mg/ml stock dissolved in PBS, filter sterilized and stored protected from light at 4° C.) was then added to each well and incubated for 2 hr. The fluorescence was recorded using a multiwell plate reader (Perkin Elmer) at Ex. 560 nm and Em. 610 nm.

### Experimental Results

### Identification of Potent High Affinity Target Binding Peptides

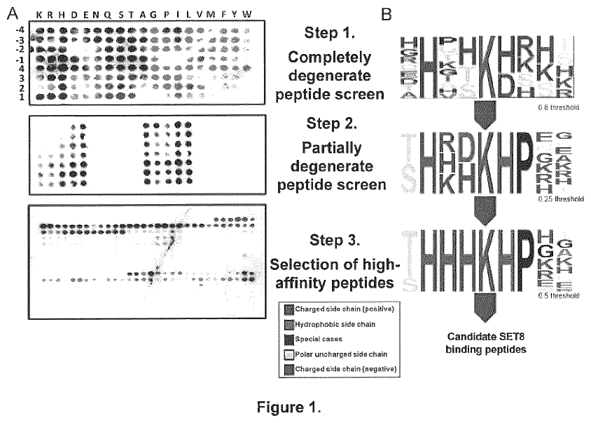

A high affinity peptide screen was carried out against target protein Set8. The method involves the sequential synthesis and printing of OPALs. FIG. 1 shows the binding of Set8 to the unselective degenerate peptide arrays. The intensity of dark spots represents the binding affinity which is quantified by ImageJ protein array analyzer.

Further the best hits from the arrays were then used to design sequence-selective peptides (FIG. 1) followed next by the sequence-specific high affinity peptides (FIG. 1). At the end of the experiment, we have successfully selected 50 Set8 specific (Table below) potential high affinity peptides.

### Differential Scanning Fluorimetry (DSF)

A total of 50 Set8 inhibitors selected from OPAL screening were synthesized on solid phase on a ResPep SL peptide synthesizer following the Fmoc chemistry protocol. All the potential inhibitors were subjected to DSF screen. A remarkable thermal shift of 4-15° C. with SYPRO Orange staining was observed for 15 peptides, KBL1-15. The data suggests that the Set8-inhibitor adducts are more stable to thermal denaturation. It remains interesting to solve the structure of the inhibitor-bound Set8 to further elucidate the mode of interaction.

### In Vitro Validation: KMTase Inhibition

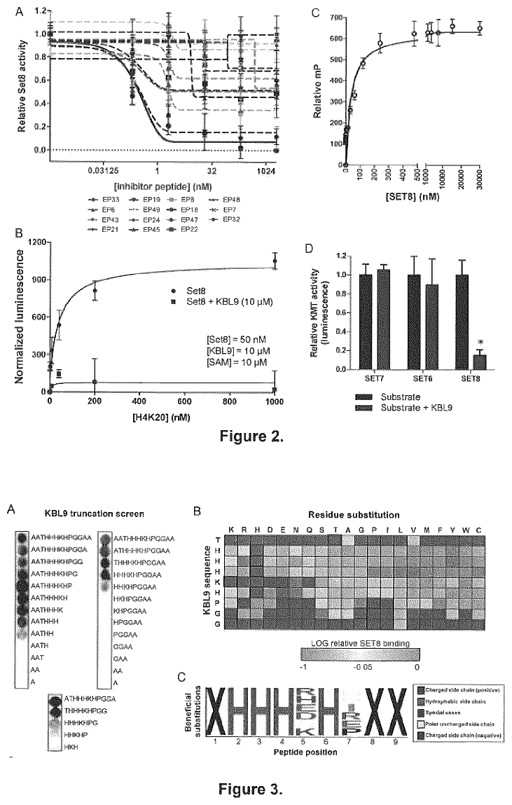

Select lysine-centered Set8 inhibitors from DSF screen were tested for in vitro inhibition of H4K20 methylation using MTAse Glo assay (Promega) as described in the materials and methods section (FIG. 2A). Out of all these inhibitors KBL9 (i.e., EP18) was found to be the most consistent and robust. A Set8:KBL9 dose-response inhibition experiment was performed, showing complete inhibition of Set8 methyltransferase activity in vitro (FIG. 2B). In order to quantify the dissociation kinetics of KBL9 with Set8, fluorescent polarization was performed, and it was determined that KBL9 bound to Set8 with an experimental Kd of 33.5+1-6.5 nM (FIG. 2C). KBL9 was further shown to display in vitro specificity towards the inhibition of Set8 activity in a panel of related methyltransferases, recombinant Set7 and Set6 enzymes (FIG. 2D).

### Characterization of Critical Binding Residues of KBL9

The position and contribution of critical residues within the KBL9 inhibitor were assessed by peptide array and in vitro recombinant Set8-His6× binding assay. Progressive C-terminal, N-terminal, and tandem truncations of KBL9 sequence were used to assess the individual residue contribution of KBL9 to Set8-His6× binding. Relative binding was qualitatively determined by chemiluminescence (FIG. 3A). A systematic mutation of the KBL9 was also carried out to in order to assess possible amino acid mutations that alter in vitro Set8-His6× binding activity, either resulting in a maintenance or strengthening (green; greater than 0 LOG(relative KBL9 binding)), tolerable (yellow; −0.05 (50% percentile) of LOG(relative KBL9 binding)) or intolerable interaction (red; −1 of LOG(relative KBL9 binding)) (FIG. 3B). The position-specific tolerable mutations that can be made to KBL9 were determined by those amino acid substitutions that retained at least 100% of WT KBL9 Set8-His6× relative binding activity (FIG. 3C).

### Cellular Activity of Set8 Inhibitor

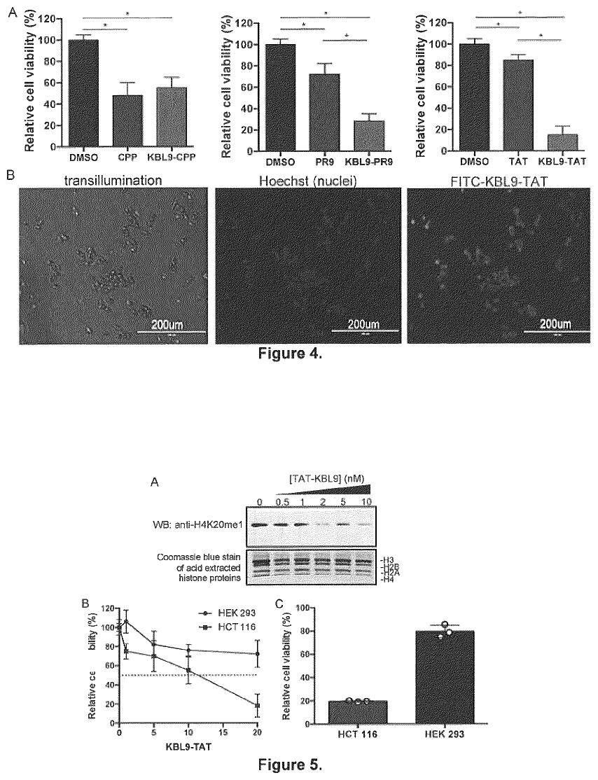

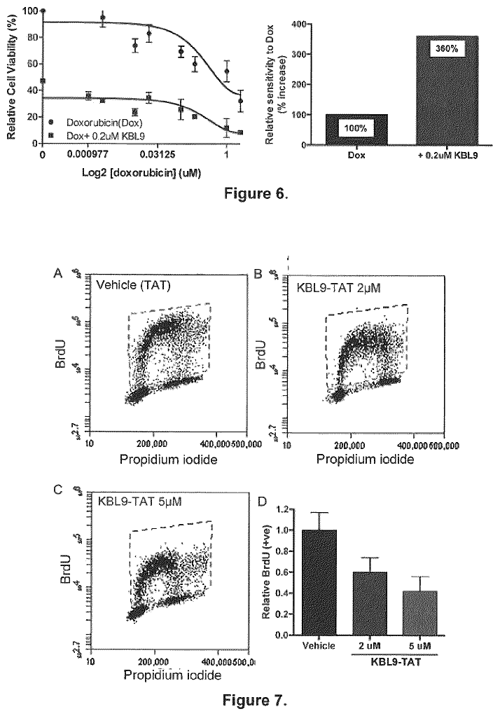

The KBL9 inhibitor was optimized for cellular delivery through the use of conjugated cell penetrating peptides (FIG. 4A). TAT-KBL9 showed the most consistent loss of HCT 116 cell viability up to 80% and chosen for further experiment. Cell penetrating peptide CPP by itself showed 50% loss of cell viability followed by PR9 (30-50%) whose overall performance was also low compared to the TAT-inhibitors and hence discontinued further (FIG. 4A). Next, the KBL9-TAT peptide was tagged with fluorophore FITC on the C-terminal end. 10 μM of the FITC tagged peptide after 24 hr post treatment when visualized under the fluorescent microscope shown to be successfully delivered into the nucleus which is seen as green foci (FIG. 4B).

### Set8 Inhibitor Reduced Cellular Histone H4K20 Methylation Status

HCT 116 colorectal carcinoma cell line was treated with the Set8 inhibitor TAT-KBL9 to test dynamic changes in H4K20 mono-methylation levels. Following TAT-KBL9 treatment, H4K20 histone lysine methylation decreased after treatment with at least 2 nM of the Set8 inhibitor (FIG. 5A).

The inhibitor was also tested for loss of cell viability in a dose dependent manner in both HCT 116 and HEK 293 cells. TAT-KBL9 turns out to be the most effective Set8 inhibitor with an IC₅₀ value ˜20 nM (FIG. 5B-C).

### Set8 Inhibitor Increases HCT 116 Cell Sensitivity to the Chemotherapeutic Agent, Doxorubicin

HCT 116 colorectal carcinoma cell line was pre-treated with the Set8 inhibitor TAT-KBL9 for 24 hrs to determine its ability to sensitize cells to periods of chemotherapy-induced DNA damage. 2 uM of KBL9-TAT was found to increase the sensitivity of HCT 116 cells to doxorubicin treatment by 360% when normalized to TAT-alone (delivery vector) treatment conditions.

### Set8 Inhibitor Decreases DNA Replication in Treated HCT 116 Cells

To help elucidate the cellular mechanism supporting the cellular function of KBL9, flow cytometry was used to document the effect of KBL9-TAT on DNA replication. DNA replication was monitored through the incorporation of BrdU into newly replicated DNA. It was found that 24 hr KBL9-TAT treatment decreased BrdU incorporation in a dose-responsive manner, indicating possible decreased DNA replication rates.

TABLE
Set8 inhibitors screened from OPAL array.

| | Experimental | | Selected |
|---|---|---|---|
| SEQ ID NO: | Peptide | Sequence | Peptide |
| 1 | EP 1 | THHHKHPHA | |
| 2 | EP 2 | THHHKHPHH | KBL6 |
| 3 | EP 3 | THHHKHPKH | |
| 4 | EP 4 | THHHKHPHT | |
| 5 | EP 5 | THHHKHPKT | |
| 6 | EP 6 | THHHKHPHK | KBL2 |
| 7 | EP 7 | THHHKHPEQ | KBL13 |
| 8 | EP 8 | THHHKHPHQ | KBL15 |
| 9 | EP 9 | SHHHKHPKA | |
| 10 | EP 10 | THHHKHPHE | |
| 11 | EP 11 | THHHKHPGT | |
| 12 | EP 12 | THHHKHPEG | |
| 13 | EP 13 | THHHKHPGH | |
| 14 | EP 14 | THHHKHPHG | |
| 15 | EP 15 | THHHKHPKA | |
| 16 | EP 16 | THHHKHPGK | |
| 17 | EP 17 | THHHKHPKK | |
| 18 | EP 18 | THHHKHPGG | KBL9 |
| 19 | EP 19 | THHHKHPGQ | KBL8 |
| 20 | EP 20 | SHHHKHPGT | |
| 21 | EP 21 | THHHKHPGR | KBL4 |
| 22 | EP 22 | THHHKHPGA | KBL6 |
| 23 | EP 23 | THHHKHPGE | |
| 24 | EP 24 | SHHHKHPHG | KBL10 |
| 25 | EP 25 | SHHHKHPGH | |
| 26 | EP 26 | THHHnHPHA | |
| 27 | EP 27 | THHHnHPHH | |
| 28 | EP 28 | THHHnHPKH | |
| 29 | EP 29 | THHHnHPHT | |
| 30 | EP 30 | THHHnHPKT | |
| 31 | EP 31 | THHHnHPHK | |
| 32 | EP 32 | THHHnHPEQ | KBL14 |
| 33 | EP 33 | THHHnHPHQ | KBL1 |
| 34 | EP 34 | SHHHnHPKA | |
| 35 | EP 35 | THHHnHPHE | |
| 36 | EP 36 | THHHnHPGT | |
| 37 | EP 37 | THHHnHPEG | |
| 38 | EP 38 | THHHnHPGH | |
| 39 | EP 39 | THHHnHPHG | |
| 40 | EP 40 | THHHnHPKA | |
| 41 | EP 41 | THHHnHPGK | |
| 42 | EP 42 | THHHnHPKK | |
| 43 | EP 43 | THHHnHPGG | KBL3 |
| 44 | EP 44 | THHHnHPGQ | |
| 45 | EP 45 | SHHHnHPGT | KBL7 |
| 46 | EP 46 | THHHnHPGR | |
| 47 | EP 47 | THHHnHPGA | KBL5 |
| 48 | EP 48 | THHHnHPGE | KBL11 |
| 49 | EP 49 | SHHHnHPHG | KBL12 |
| 50 | EP 50 | SHHHnHPGH | |

n = norLeucine (Nle)

TABLE
List of the top-selected Set8 inhibitors.

| Name | Sequence |
|---|---|
| KBL1 | THHHnHPHQ |
| KBL2 | THHHKHPHK |
| KBL3 | THHHnHPGG |
| KBL4 | THHHKHPGR |
| KBL5 | THHHnHPGA |
| KBL6 | THHHKHPGA |
| KBL7 | SHHHnHPGT |
| KBL8 | THHHKHPGQ |
| KBL9 | THHHKHPGG |
| KBL10 | SHHHKHPHG |
| KBL11 | THHHnHPGE |
| KBL12 | SHHHnHPHG |
| KBL13 | THHHKHPEQ |
| KBL14 | THHHnHPEQ |
| KBL15 | THHHKHPHQ |

n = norLeucine (Nle)

TABLE
Tm shift of recombinant Set8
induced by Set8-inhibitor by DSF.

| Inhibitor | Tm (° C.) |
|---|---|
| DMSO | 71.5 ± 1.1 |
| KBL1 | 86.5 ± 0.8 |
| KBL2 | 85.8 ± 1.1 |
| KBL3 | 85.6 ± 0.6 |
| KBL4 | 85.0 ± 1.3 |
| KBL5 | 85.0 ± 0.7 |
| KBL6 | 84.8 ± 1.1 |
| KBL7 | 84.8 ± 1.2 |
| KBL8 | 84.5 ± 0.8 |
| KBL9 | 83.5 ± 0.8 |
| KBL10 | 82.8 ± 1.8 |
| KBL11 | 78.5 ± 4.0 |
| KBL12 | 78.3 ± 6.3 |
| KBL13 | 77.6 ± 7.5 |
| KBL14 | 81.5 ± 1.0 |
| KBL15 | 75.5 ± 5.3 |

### REFERENCES

Arrowsmith C H, Bountra C, Fish P V, Lee K, Schapira M. Epigenetic protein families: a new frontier for drug discovery. Nat. Rev. Drug Discov. 2012; 11, 384-400.
Beck-Sickinger A G, WWI K. Posttranslational Modification of Proteins. Expanding Nature's Inventory. By Christopher T. Walsh. Angew. Chem. Int. Ed. 2006; 45, 1020-1020.
Biggar K K, Li S S C. Non-histone protein methylation as a regulator of cellular signaling and function. Nat. Rev. Mol. Cell Biol. 2015; 16, 5-17.
Blum G, Ibanez G, Rao X, Shum D, Radu C, Djaballah H, et al. Small-molecule inhibitors of Set8 with cellular activity. ACS Chem. Biol. 2014; 9:2471-2478.
Dhami G K, Liu H, Galka M, Voss C, Wei R, Muranko K, et al. Dynamic methylation of numb by Set8 regulates its binding to p53 and apoptosis. Mol Cell. 2013; 50(4):565-76.
Ding C, Li R, Peng J, Li S, Guo Z. A polymorphism at the miR-502 binding site in the 3′ untranslated region of the Set8 gene is associated with the outcome of small-cell lung cancer. Experimental and therapeutic medicine. 2012; 3(4):689-92.
Guo Z, Wu C, Wang X, Wang C, Zhang R, Shan B. A polymorphism at the miR-502 binding site in the 3′-untranslated region of the histone methyltransferase Set8 is associated with hepatocellular carcinoma outcome. Int J Cancer. 2012; 131(6):1318-22.
Hamamoto R, Saloura V, Nakamura Y. Critical roles of non-histone protein lysine methylation in human tumorigenesis. Nat. Rev. Cancer 2015; 15, 110-124.
Hashemi M, Sheybani-Nasab M, Naderi M, Roodbari F, Taheri M. Association of functional polymorphism at the miR-502-binding site in the 3′ untranslated region of the Set8 gene with risk of childhood acute lymphoblastic leukemia, a preliminary report. Tumor Biol. 2014; 35(10): 10375-9.
Ji X, Jin S, Qu X, Li K, Wang H, He F, et al. Lysine-specific demethylase 5C promotes hepatocellular carcinoma cell invasion through inhibition BMP7 expression, BMC Cancer 2015; 26: 801.
Jin H, Zangar R C. Protein modifications as potential biomarkers in breast cancer. Biomark. Insights 2009; 4, 191-200.
Rau R C, Dou Y. Hijacked in cancer: the KMT2(MLL) family of methyltransferases. Nat. Rev. Cancer 2015; 15, 334-346.
Seo J, Lee K J. Post-translational modifications and their biological functions: proteomic analysis and systematic approaches. J. Biochem. Mol. Biol. 2014; 37, 35-44.
Shi X, Kachirskaia I, Yamaguchi H, West L E, Wen H, Wang E W, et al. Modulation of p53 function by Set8-mediated methylation at lysine 382. Mol Cell. 2007; 27(4):636-46.
Song F, Zheng H, Liu B, Wei S, Dai H, Zhang L, et al. An miR-502-binding site single-nucleotide polymorphism in the 3′-untranslated region of the Set8 gene is associated with early age of breast cancer onset. Clin Cancer Res. 2009,15(19):6292-300.
Stein J, Majores M, Rohde M, Lim S, Schneider S, Krappe E, et al. KDM5C is overexpressed in prostate cancer and is a prognostic marker for prostate-specific antigen-relapse following radical prostatectomy. Am. J. Pathol. 2014; 184: 2430-2437.
Takawa M, Cho H-S, Hayami S, Toyokawa G, Kogure M, Yamane Y, et al. Histone lysine methyltransferase Set8 promotes carcinogenesis by deregulating PCNA expression. Cancer Res. 2012; 72(13):3217-27.
Valente S, Lepore I, Dell'Aversana C, Tardugno M, Castellano S, Sbardella G, et al. Identification of PR-SET7 and EZH2 selective inhibitors inducing cell death in human leukemia U937 cells. Biochimie. 2012; 94(11):2308-13.
Veschi V, Liu Z, Voss T C, Ozbun L, Gryder B, Yan C, et al. Epigenetic siRNA and chemical screens identify Set8 inhibition as a therapeutic strategy for p53 activation in high-risk neuroblastoma. Cancer Cell 2017; 31:50-63.
Wang C, Guo Z, Wu C, Li Y, Kang S. A polymorphism at the miR-502 binding site in the 3′ untranslated region of the Set8 gene is associated with the risk of epithelial ovarian cancer. Cancer Genetics. 2012; 205(7-8): 373-6.
Wang Q, Wei J, Su P, Gao P. Histone demethylase JARID1C promotes breast cancer metastasis cells via down regulating BRMS1 expression, Biochem. Biophys. Res. Commun. 2015; 464: 659-666.
Xu J, Yin Z, Gao W, Liu L, Yin Y, Liu P, et al. Genetic variation in a microRNA-502 minding site in Set8 gene confers clinical outcome of non-small cell lung cancer in a Chinese population. PLoS One. 2013; 8(10):e77024.
Xu L, Wu W, Cheng G, Qian M, Hu K, Yin G, et al. Enhancement of proliferation and invasion of gastric cancer cell by KDM5C via decrease in p53 expression. Technol. Cancer Res. Treat. 2017; 16:141-149.
Yang F, Sun L, Li Q, Han X, Lei L, Zhang H, et al. Set8 promotes epithelial mesenchymal transition and confers TWIST dual transcriptional activities. EMBO J. 2012; 31(1):110-23.
Yao L, Li Y, Du F, Han X, Li X, Niu Y, et al. Histone H4 Lys 20 methyltransferase Set8 promotes androgen receptor-mediated transcription activation in prostate cancer. Biochem Biophys Res Commun. 2014; 450(1):692-6.
Zhang X, Wen H, Shi X. Lysine methylation: beyond histones. Acta Biochim. Biophys. Sin. 2012; 44, 14-27.

1. A peptide that binds to Set8.

2. A peptide that binds to Set8, wherein said peptide comprises the sequence:
X₂X₃X₄X₅X₆X₇X₈X₉; where
X₁=T or S
X₂=H
X₃=H
X₄=H
X₅=K or Nle
X₆=H
X₇=P
X₈=G, H or E
X₉=Q, K, G, R, A, T or E; or a binding fragment thereof

3. A peptide that binds to Set8 and comprises the sequence selected from the group consistingofTHHHKHPHA; THHHKHPHH; THHHKHPKH; THHHKHPH T; THHHKHPKT; THHHKHPHK; THHHKHPEQ; THHHKHPHQ; SHH HKHPKA; THHHKHPHE; THHHKHPGT; THHHKHPEG; THHHKHP GH; THHHKHPHG; THHHKHPKA; THHHKHPGK; THHHKHPKK; T HHHKHPGG; THHHKHPGQ; SHHHKHPGT; THHHKHPGR; THHHK HPGA; THHHKHPGE; SHHHKHPHG; SHHHKHPGH; THHHnHPHA; THHHnHPHH; THHHnHPKH; THHHnHPHT; THHHnHPKT; THHHn HPHK; THHHnHPEQ; THHHnHPHQ; SHHHnHPKA; THHHnHPHE; THHHnHPGT; THHHnHPEG; THHHnHPGH; THHHnHPHG; THHHn HPKA; THHHnHPGK; THHHnHPKK; THHHnHPGG; THHHnHPGQ; S HHHnHPGT; THHHnHPGR; THHHnHPGA; THHHnHPGE; SHHHnH PHG; and SHHHnHPGH; wherein n=norLeucine (Nle) or a binding fragment thereof.

4. The peptide of any one of claims 1 to 3, further comprising a cell penetrating peptide.

5. The peptide of claim 4, wherein said cell penetrating peptide is a TAT cell penetrating peptide.

6. The peptide of claim 4 or 5, wherein said cell penetrating peptide is attached via a 6-aminohexanoic acid linker.

7. A peptide comprising the sequence selected from the group consisting of:
THHHnHPHQ{6-aminohexanoic acid}GRKKRRQRRRPPQ;
THHHKHPHK{6-aminohexanoic acid}GRKKRRQRRRPPQ;
THHHnHPGG{6-aminohexanoic acid}GRKKRRQRRRPPQ;
THHHKHPGR{6-aminohexanoic acid}GRKKRRQRRRPPQ;
THHHnHPGA{6-aminohexanoic acid}GRKKRRQRRRPPQ;
THHHKHPGA{6-aminohexanoic acid}GRKKRRQRRRPPQ;
SHHHnHPGT{6-aminohexanoic acid}GRKKRRQRRRPPQ;
THHHKHPGQ{6-aminohexanoic acid}GRKKRRQRRRPPQ;
THHHKHPGG{6-aminohexanoic acid}GRKKRRQRRRPPQ;
SHHHKHPHG{6-aminohexanoic acid}GRKKRRQRRRPPQ;
THHHnHPGE{6-aminohexanoic acid}GRKKRRQRRRPPQ;
SHHHnHPHG{6-aminohexanoic acid}GRKKRRQRRRPPQ;
THHHKHPEQ{6-aminohexanoic acid}GRKKRRQRRRPPQ;
THHHnHPEQ{6-aminohexanoic acid}GRKKRRQRRRPPQ;
THHHKHPHQ{6-aminohexanoic acid}GRKKRRQRRRPPQ;
THHHnHPHQ{6-aminohexanoic acid}FFLIPKGRRRRRRRRR;
THHHKHPHK{6-aminohexanoic acid}FFLIPKGRRRRRRRRR;
THHHnHPGG{6-aminohexanoic acid}FFLIPKGRRRRRRRRR;
THHHKHPGR{6-aminohexanoic acid}FFLIPKGRRRRRRRRR;
THHHnHPGA{6-aminohexanoic acid}FFLIPKGRRRRRRRRR;
THHHKHPGA{6-aminohexanoic acid}FFLIPKGRRRRRRRRR;
SHHHnHPGT{6-aminohexanoic acid}FFLIPKGRRRRRRRRR;
THHHKHPGQ{6-aminohexanoic acid}FFLIPKGRRRRRRRRR;
THHHKHPGG{6-aminohexanoic acid}FFLIPKGRRRRRRRRR;
SHHHKHPHG{6-aminohexanoic acid}FFLIPKGRRRRRRRRR;
THHHnHPGE{6-aminohexanoic acid}FFLIPKGRRRRRRRRR;
SHHHnHPHG{6-aminohexanoic acid}FFLIPKGRRRRRRRRR;
THHHKHPEQ{6-aminohexanoic acid}FFLIPKGRRRRRRRRR;
THHHnHPEQ{6-aminohexanoic acid}FFLIPKGRRRRRRRRR;
THHHKHPHQ{6-aminohexanoic acid}FFLIPKGRRRRRRRRR;
THHHnHPHQ{6-aminohexanoic acid}RRWRRWRRWRR;
THHHKHPHK{6-aminohexanoic acid}RRWRRWRRWRR;
THHHnHPGG{6-aminohexanoic acid}RRWRRWRRWRR;
THHHKHPGR{6-aminohexanoic acid}RRWRRWRRWRR;
THHHnHPGA{6-aminohexanoic acid}RRWRRWRRWRR;
THHHKHPGA{6-aminohexanoic acid}RRWRRWRRWRR;
SHHHnHPGT{6-aminohexanoic acid}RRWRRWRRWRR;
THHHKHPGQ{6-aminohexanoic acid}RRWRRWRRWRR;
THHHKHPGG{6-aminohexanoic acid}RRWRRWRRWRR;
SHHHKHPHG{6-aminohexanoic acid}RRWRRWRRWRR;
THHHnHPGE{6-aminohexanoic acid}RRWRRWRRWRR;
SHHHnHPHG{6-aminohexanoic acid}RRWRRWRRWRR;
THHHKHPEQ{6-aminohexanoic acid}RRWRRWRRWRR;
THHHnHPEQ{6-aminohexanoic acid}RRWRRWRRWRR;
and
THHHKHPHQ{6-aminohexanoic acid}RRWRRWRRWRR.

8. A peptide that binds to Set8, wherein said peptide comprises the sequence:
X₁=any amino acid
X₂=H
X₃=H
X₄=H
X₅=R, H, E, D, Q, K
X₆=H, E
X₇=P, N, Q, I, R, E
X₈=M, N, E, D, Q, P, F, W, K, I, G, V
X₉=any amino acid or a bind fragment thereof.

9. The peptide of any one of claims 1 to 8, wherein said peptide inhibits Set8 activity.

10. A polynucleotide encoding one or more peptides of any one of claims 1, 2, 3 and 8.

11. A vector comprising the polynucleotide of claim 10.

12. A pharmaceutical composition comprising one or more peptides of any one of claims 1 to 9, one or more polynucleotides of claim 10 or one or more vectors of claim 11 and a pharmaceutically acceptable carrier.

13. A method of inhibiting the activity of Set8 in a subject in need thereof, comprising administering one or more of the peptides of any one of claims 1 to 9, one or more polynucleotides of claim 10, one or more vectors of claim 11 or the pharmaceutical composition of claim 12.

14. A method of treating a disease associate with increased Set8 in a subject in need thereof, comprising administering one or more of the peptides of any one of claims 1 to 9, one or more polynucleotides of claim 10, one or more vectors of claim 11 or the pharmaceutical composition of claim 12.

15. The method of claim 14, wherein the disease is cancer.


SEQUENCE LISTING (72 sequences)

SEQ ID NO: 1 - protein, 9 aa, Artificial Sequence
  peptide binding to SET8
THHHKHPHA

SEQ ID NO: 2 - protein, 9 aa, Artificial Sequence
  petide binding to SET8
THHHKHPHH

SEQ ID NO: 3 - protein, 9 aa, Artificial Sequence
  peptide binding to SET8
THHHKHPKH

SEQ ID NO: 4 - protein, 9 aa, Artificial Sequence
  peptide binding to SET8
THHHKHPHT

SEQ ID NO: 5 - protein, 9 aa, Artificial Sequence
  peptide binding to SET8
THHHKHPKT

SEQ ID NO: 6 - protein, 9 aa, Artificial Sequence
  peptide binding to SET8
THHHKHPHK

SEQ ID NO: 7 - protein, 9 aa, Artificial Sequence
  peptide binding to SET8
THHHKHPEQ

SEQ ID NO: 8 - protein, 9 aa, Artificial Sequence
  peptide binding to SET8
THHHKHPHQ

SEQ ID NO: 9 - protein, 9 aa, Artificial Sequence
  peptide binding to SET8
SHHHKHPKA

SEQ ID NO: 10 - protein, 9 aa, Artificial Sequence
  peptide binding to SET8
THHHKHPHE

SEQ ID NO: 11 - protein, 9 aa, Artificial Sequence
  peptide binding to SET8
THHHKHPGT

SEQ ID NO: 12 - protein, 9 aa, Artificial Sequence
  peptide binding to SET8
THHHKHPEG

SEQ ID NO: 13 - protein, 9 aa, Artificial Sequence
  peptide binding to SET8
THHHKHPGH

SEQ ID NO: 14 - protein, 9 aa, Artificial Sequence
  peptide binding to SET8
THHHKHPHG

SEQ ID NO: 15 - protein, 9 aa, Artificial Sequence
  peptide binding to SET8
THHHKHPKA

SEQ ID NO: 16 - protein, 9 aa, Artificial Sequence
  peptide binding to SET8
THHHKHPGK

SEQ ID NO: 17 - protein, 9 aa, Artificial Sequence
  peptide binding to SET8
THHHKHPKK

SEQ ID NO: 18 - protein, 9 aa, Artificial Sequence
  peptide binding to SET8
THHHKHPGG

SEQ ID NO: 19 - protein, 9 aa, Artificial Sequence
  peptide binding to SET8
THHHKHPGQ

SEQ ID NO: 20 - protein, 9 aa, Artificial Sequence
  peptide binding to SET8
SHHHKHPGT

SEQ ID NO: 21 - protein, 9 aa, Artificial Sequence
  peptide binding to SET8
THHHKHPGR

SEQ ID NO: 22 - protein, 9 aa, Artificial Sequence
  peptide binding to SET8
THHHKHPGA

SEQ ID NO: 23 - protein, 9 aa, Artificial Sequence
  peptide binding to SET8
THHHKHPGE

SEQ ID NO: 24 - protein, 9 aa, Artificial Sequence
  peptide binding to SET8
SHHHKHPHG

SEQ ID NO: 25 - protein, 9 aa, Artificial Sequence
  peptide binding to SET8
SHHHKHPGH

SEQ ID NO: 26 - protein, 9 aa, Artificial Sequence
  peptide binding to SET8
  MISC_FEATURE (5)..(5): norLeucine (Nle)
THHHXHPHA

SEQ ID NO: 27 - protein, 9 aa, Artificial Sequence
  peptide binding to SET8
  MISC_FEATURE (5)..(5): norLeucine (Nle)
THHHXHPHH

SEQ ID NO: 28 - protein, 9 aa, Artificial Sequence
  peptide binding to SET8
  MISC_FEATURE (5)..(5): norLeucine (Nle)
THHHXHPKH

SEQ ID NO: 29 - protein, 9 aa, Artificial Sequence
  peptide binding to SET8
  MISC_FEATURE (5)..(5): norLeucine (Nle)
THHHXHPHT

SEQ ID NO: 30 - protein, 9 aa, Artificial Sequence
  peptide binding to SET8
  MISC_FEATURE (5)..(5): norLeucine (Nle)
THHHXHPKT

SEQ ID NO: 31 - protein, 9 aa, Artificial Sequence
  peptide binding to SET8
  MISC_FEATURE (5)..(5): norLeucine (Nle)
THHHXHPHK

SEQ ID NO: 32 - protein, 9 aa, Artificial Sequence
  peptide binding to SET8
  MISC_FEATURE (5)..(5): norLeucine (Nle)
THHHXHPEQ

SEQ ID NO: 33 - protein, 9 aa, Artificial Sequence
  peptide binding to SET8
  MISC_FEATURE (5)..(5): norLeucine (Nle)
THHHXHPHQ

SEQ ID NO: 34 - protein, 9 aa, Artificial Sequence
  peptide binding to SET8
  MISC_FEATURE (5)..(5): norLeucine (Nle)
SHHHXHPKA

SEQ ID NO: 35 - protein, 9 aa, Artificial Sequence
  peptide binding to SET8
  MISC_FEATURE (5)..(5): norLeucine (Nle)
THHHXHPHE

SEQ ID NO: 36 - protein, 9 aa, Artificial Sequence
  peptide binding to SET8
  MISC_FEATURE (5)..(5): norLeucine (Nle)
THHHXHPGT

SEQ ID NO: 37 - protein, 9 aa, Artificial Sequence
  peptide binding to SET8
  MISC_FEATURE (5)..(5): norLeucine (Nle)
THHHXHPEG

SEQ ID NO: 38 - protein, 9 aa, Artificial Sequence
  peptide binding to SET8
  MISC_FEATURE (5)..(5): norLeucine (Nle)
THHHXHPGH

SEQ ID NO: 39 - protein, 9 aa, Artificial Sequence
  peptide binding to SET8
  MISC_FEATURE (5)..(5): norLeucine (Nle)
THHHXHPHG

SEQ ID NO: 40 - protein, 9 aa, Artificial Sequence
  peptide binding to SET8
  MISC_FEATURE (5)..(5): norLeucine (Nle)
THHHXHPKA

SEQ ID NO: 41 - protein, 9 aa, Artificial Sequence
  peptide binding to SET8
  MISC_FEATURE (5)..(5): norLeucine (Nle)
THHHXHPKA

SEQ ID NO: 42 - protein, 9 aa, Artificial Sequence
  peptide binding to SET8
  MISC_FEATURE (5)..(5): norLeucine (Nle)
THHHXHPKK

SEQ ID NO: 43 - protein, 9 aa, Artificial Sequence
  peptide binding to SET8
  MISC_FEATURE (5)..(5): norLeucine (Nle)
THHHXHPGG

SEQ ID NO: 44 - protein, 9 aa, Artificial Sequence
  peptide binding to SET8
  MISC_FEATURE (5)..(5): norLeucine (Nle)
THHHXHPGQ

SEQ ID NO: 45 - protein, 9 aa, Artificial Sequence
  peptide binding to SET8
  MISC_FEATURE (5)..(5): norLeucine (Nle)
SHHHXHPGT

SEQ ID NO: 46 - protein, 9 aa, Artificial Sequence
  peptide binding to SET8
  MISC_FEATURE (5)..(5): norLeucine (Nle)
THHHXHPGR

SEQ ID NO: 47 - protein, 9 aa, Artificial Sequence
  peptide binding to SET8
  MISC_FEATURE (5)..(5): norLeucine (Nle)
THHHXHPGA

SEQ ID NO: 48 - protein, 9 aa, Artificial Sequence
  peptide binding to SET8
  MISC_FEATURE (5)..(5): norLeucine (Nle)
THHHXHPGE

SEQ ID NO: 49 - protein, 9 aa, Artificial Sequence
  peptide binding to SET8
  MISC_FEATURE (5)..(5): norLeucine (Nle)
SHHHXHPHG

SEQ ID NO: 50 - protein, 9 aa, Artificial Sequence
  peptide binding to SET8
  MISC_FEATURE (5)..(5): norLeucine (Nle)
SHHHXHPGH

SEQ ID NO: 51 - protein, 13 aa, Artificial Sequence
  TAT48-60
GRKKRRQRRRPPQ

SEQ ID NO: 52 - protein, 9 aa, Artificial Sequence
  TAT49-57
RKKRRQRRR

SEQ ID NO: 53 - protein, 16 aa, Artificial Sequence
  Penetratin, pAntp(43-58)
RQIKIWFQNRRMKWKK

SEQ ID NO: 54 - protein, 19 aa, Artificial Sequence
  DPV1047
VKRGLKLRHVRPRVTRMDV

SEQ ID NO: 55 - protein, 16 aa, Artificial Sequence
  PR9
FFLIPKGRRRRRRRRR

SEQ ID NO: 56 - protein, 11 aa, Artificial Sequence
  Mut6DPT (CPP)
RRWRRWRRWRR

SEQ ID NO: 57 - protein, 27 aa, Artificial Sequence
  MPG
GALFLGFLGAAGSTMGAWSQPKKKRKV

SEQ ID NO: 58 - protein, 21 aa, Artificial Sequence
  Pep-1
KETWWETWWTEWSQPKKKRKV

SEQ ID NO: 59 - protein, 18 aa, Artificial Sequence
  pVEC
LLIILRRRIRKQAHAHSK

SEQ ID NO: 60 - protein, 22 aa, Artificial Sequence
  ARF(1-22)
MVRRFLVTLRIRRACGPPRVRV

SEQ ID NO: 61 - protein, 28 aa, Artificial Sequence
  BPrPr(1-28)
MVKSKIGSWILVLFVAMWSDVGLCKKRP

SEQ ID NO: 62 - protein, 18 aa, Artificial Sequence
  MAP
KLALKLALKALKAALKLA

SEQ ID NO: 63 - protein, 27 aa, Artificial Sequence
  Transportan
GWTLNSAGYLLGKINLKALAALAKKIL

SEQ ID NO: 64 - protein, 28 aa, Artificial Sequence
  p28
LSTAADMQGVVTDGMASGLDKDYLKPDD

SEQ ID NO: 65 - protein, 26 aa, Artificial Sequence
  VT5
DPKGDPKGVTVTVTVTVTGKGDPKPD

SEQ ID NO: 66 - protein, 24 aa, Artificial Sequence
  Bac 7 (Bac 1-24)
RRIRPRPPRLPRPRPRPLPFPRPG

SEQ ID NO: 67 - protein, 17 aa, Artificial Sequence
  C105Y
CSIPPEVKFNKPFVYLI

SEQ ID NO: 68 - protein, 6 aa, Artificial Sequence
  PFVYLI
PFVYLI

SEQ ID NO: 69 - protein, 15 aa, Artificial Sequence
  Pep-7
SDLWEMMMVSLACQY

SEQ ID NO: 70 - protein, 162 aa, Artificial Sequence
  Set8
AAIAKQALKKPIKGKQAPRKKAQGKTQQNRKLTDFYPVRRSSRKSKAELQSEERKRIDELIESGKEEGMKIDLIDGKGRGVIATKQFSRGDFVVEYHGDLIEITDAKKREALYAQDPSTGCYMYYFQYLSKTYCVDATRETNRLGRLINHSKCGNCQTKLHD

SEQ ID NO: 71 - protein, 9 aa, artificial sequence
  consensus sequence for SET8 binding peptide
  misc_feature (1)..(1): Thr or Ser
  misc_feature (5)..(5): Lys or Nle
  misc_feature (8)..(8): Gly, His or Glu
  misc_feature (9)..(9): Gln, Lys, Gly, Arg, Ala, Thr or Glu
XHHHXHPXX

SEQ ID NO: 72 - protein, 9 aa, artificial sequence
  peptide binding SET8
  misc_feature (1)..(1): any amino acid
  misc_feature (5)..(5): Arg, His, Glu, Asp, Gln or Lys
  misc_feature (6)..(6): His or Glu
  misc_feature (7)..(7): Pro, Asn, Gln, Ile, Arg or Glu
  misc_feature (8)..(8): Met, Asn, Glu, Asp, Gln, Pro, Phe, Trp, Lys, Ile, Gly or Val
  misc_feature (9)..(9): Xaa can be any naturally occurring amino acid
XHHHXXXXX